(19) United States
(12) Patent Application Publication
(10) Pub. No.: US 2023/0364342 A1
(43) Pub. Date: Nov. 16, 2023

(54) PRE-FILLED MULTI-FLUID MEDICAL DELIVERY ASSEMBLIES

(71) Applicant: Koska Family Limited, East Sussex (GB)

(72) Inventors: Marc Andrew Koska, Eastbourne (GB); Max Hannon, Flemingate (UA); Harris Ryder, Cambrose (UA)

(21) Appl. No.: 18/221,602

(22) Filed: Jul. 13, 2023

Related U.S. Application Data
(63) Continuation of application No. PCT/IB2022/051429, filed on Feb. 17, 2022.
(60) Provisional application No. 63/154,280, filed on Feb. 26, 2021.

(51) Int. Cl. A61M 5/28; A61M 39/10

(52) U.S. Cl. CPC A61M 5/284; A61M 39/10

(57) ABSTRACT

A pre-filled medical delivery assembly can have first and second blow-fill-seal (BFS) modules and first and second connectors. Each BFS module can have a reservoir with a fluid agent and a neck with a seal. Each connector can have a respective recess with a piercing element. The first connector can have an outlet port, and the second connector can have an inlet/outlet (I/O) port. The connectors can be coupled together via the outlet and I/O ports. The neck of each BFS module can be inserted into the recess of the respective connector such that the piercing element breaches the seal, thereby providing a fluid path between the reservoirs. The disclosed assemblies can allow the combination of separate fluid agents via the coupled connectors and subsequently delivery of the combination as a single dose of a therapeutic agent to a patient by replacing the first connector with an administration hub.

### CROSS-REFERENCE TO RELATED APPLICATIONS

This application is a Continuation Application of International PCT Application No. PCT/IB2022/051429, filed on Feb. 17, 2022 in the name of Koska et al. and titled PRE-FILLED MULTI-FLUID MEDICAL DELIVERY ASSEMBLIES, which PCT Application claims benefit of and priority under 35 U.S.C. § 119(e) to, and is a Non-provisional of U.S. Provisional Patent Application No. 63/154,280 filed on Feb. 26, 2021 and titled “PRE-FILLED DUAL-LIQUID MEDICAL DELIVERY ASSEMBLIES.” Each of these Applications is hereby incorporated by reference herein in its entirety and for all purposes.

### STATEMENT REGARDING FEDERALLY SPONSORED RESEARCH

This invention was made with government support under Award No. 75A50120C00007, awarded by the Department of Health and Human Services (HHS). The government has certain rights in the invention.

### BACKGROUND

Every year, millions of people become infected and die from a variety of diseases, some of which are vaccine-preventable. Although vaccination has led to a dramatic decline in the number of cases of several infectious diseases, some of these diseases remain quite common. In many instances, large populations of the world, particularly in developing countries, suffer from the spread of vaccine-preventable diseases due to ineffective immunization programs, either because of poor implementation, lack of affordable vaccines, or inadequate devices for administering vaccines, or combinations thereof.

Some implementations of immunization programs include administration of vaccines via a reusable syringe. However, in many situations, particularly in developing countries, the administration of vaccines occur outside of a hospital and may be provided by a non-professional, such that injections are given to patients without carefully controlling access to syringes. The use of reusable syringes under those circumstances increases the risk of infection and spread of blood-borne diseases, particularly when syringes, which have been previously used and are no longer sterile, are used to administer subsequent injections. For example, the World Health Organization (WHO) estimates that blood-borne diseases, such as Hepatitis and human immunodeficiency virus (HIV), are being transmitted due to reuse of such syringes, resulting in the death of more than one million people each year.

Previous attempts at providing single-use or disposable injection devices to remedy such problems in the industry have achieved measurable success but have failed to adequately remedy the existing problems. Pre-filled, single-use injection devices manufactured via injection molding or Form-Fill-Seal (FFS) processes, such as the Uniject™ device available from the Becton, Dickinson and Company of Franklin Lakes, NJ, for example, while offering precise manufacturing tolerances in the range of two thousandths of an inch (0.002-in; 50.8 μm) to four thousandths of an inch (0.004-in; 101.6 μm)—for hole diameters in molded parts, require separate sterilization processes (e.g., gamma radiation) that are not compatible with certain fluids, provide production rates limited to approximately nine thousand (9,000) non-sterile units per hour, and can be provided to an end-user for approximately one dollar and forty cents ($1.40) per dose/unit. Such devices also are not configured to store, mix, and/or deliver a medicament comprising multiple liquids such as a vaccine and an adjuvant.

### SUMMARY

Embodiments of the disclosed subject matter provide systems, assemblies, kits, and methods for medical delivery of liquid agents to a patient. Using a blow-fill-seal (BFS) fabrication technique, separate BFS modules (such as vials, bottles, or other components) are pre-filled with respective fluid (e.g., liquid) agents, which can be maintained separate, for example, until ready for use or administration to a patient. In some embodiments, first and second connectors can have respective piercing elements therein and can be releasably coupled together. Part of a first BFS module can be inserted into the first connector such that the piercing element thereof breaches a seal of the first BFS module, and a part of a second BFS module can be inserted into the second connector such that the piercing element thereof breaches a seal of the second BFS module. With the first and second connectors coupled together, reservoirs of the first and second BFS modules can be fluidically connected together. Actuation (e.g., lateral or longitudinal compression) of the first BFS module dispenses a first fluid agent from the reservoir of the first BFS module via the coupled connectors into the reservoir of the second BFS module, thereby combining the first fluid agent with a second fluid agent in the second BFS module. The fluid agents from the separate reservoirs can thus be combined prior to use or administration.

In some embodiments, the first connector with first BFS module (e.g., partially or fully emptied, exhausted, or spent) can be decoupled from the second connector, for example, to allow an administration assembly to be coupled to the second connector. In some embodiments, the administration assembly can comprise a needle, cannula or a nozzle. With the administration assembly and second connector coupled together, the reservoir of the second BFS module can be fluidically connected to an outlet of the administration assembly. Actuation (e.g., lateral or longitudinal compression) of the second BFS module dispenses the combined fluid agents from the second reservoir along the second flow path and through the outlet of the administration assembly. For example, embodiments of the disclosed subject matter can deliver the combination of fluid agents from the BFS module as a single dose of a therapeutic agent (e.g., vaccine, drug, medicament, etc.).

In one or more embodiments, a pre-filled medical delivery assembly can comprise a first blow-fill-seal (BFS) module, a second BFS module, a first connector, and a second connector. The first BFS module can have a first reservoir and a first neck. The first reservoir can have a first fluid agent therein. The first neck can have a first seal at an end thereof and can be in fluid communication with the first reservoir. The second BFS module can have a second reservoir and a second neck. The second reservoir can have a second fluid agent therein. The second neck can have a second seal at an end thereof and can be in fluid communication with the second reservoir. The first connector can have a first recess and an outlet port. The first recess can be constructed to receive at least part of the first neck of the first BFS module. The outlet port can be in fluid communication with the first recess. The first recess can have a first piercing element therein. The second connector can have a second recess and an inlet/outlet (I/O) port. The second recess can be constructed to receive at least part of the second neck of the second BFS module. The I/O port can be in fluid communication with the second recess. The second recess can have a second piercing element therein. The first and second connectors can be coupled together via the outlet port and the I/O port so as to provide a fluid path between the first and second reservoirs.

Any of the various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosed technology will become more apparent from the following detailed description, which proceeds with reference to the accompanying figures.

### BRIEF DESCRIPTION OF THE DRAWINGS

Where applicable, some elements may be simplified or otherwise not illustrated in order to assist in the illustration and description of underlying features. For example, in some figures, some components have been illustrated using a partial or cutaway view in order to illustrate internal interaction of components. Throughout the figures, like reference numerals denote like elements. An understanding of embodiments described herein and many of the attendant advantages thereof may be readily obtained by reference to the following detailed description when considered with the accompanying drawings, wherein:

FIG. 1 is an exploded view of components of a pre-filled medical fluid assembly, according to one or more embodiments of the disclosed subject matter;

FIG. 2A is a simplified cross-sectional view of a pre-filled medical fluid assembly prior to breach of fluid seals of a pair of blow-fill-seal (BFS) modules, according to one or more embodiments of the disclosed subject matter;

FIG. 2B is a simplified cross-sectional view of the pre-filled medical fluid assembly of FIG. 2A after dispensing of fluid from the first BFS module to the second BFS module, according to one or more embodiments of the disclosed subject matter;

FIG. 2C is a simplified cross-sectional view of the pre-filled medical fluid assembly of FIG. 2B after decoupling of the first connector from the second connector, according to one or more embodiments of the disclosed subject matter;

FIG. 2D is a simplified cross-sectional view of the pre-filled medical fluid sub-assembly of FIG. 2C prior to coupling with an administration hub, according to one or more embodiments of the disclosed subject matter;

FIG. 2E is a simplified cross-sectional view of the pre-filled medical fluid sub-assembly of FIG. 2D after coupling with the administration hub to form a medical delivery assembly, according to one or more embodiments of the disclosed subject matter;

FIG. 2F is a simplified cross-sectional view of the medical delivery assembly of FIG. 2E after removal of a needle cap in preparation for injection, according to one or more embodiments of the disclosed subject matter; and

FIG. 3 is a process flow diagram of an exemplary method for assembly and use of a pre-filled medical delivery assembly for administration of combined fluid agents, according to one or more embodiments of the disclosed subject matter.

### DETAILED DESCRIPTION

### I. Introduction

Described herein are systems, assemblies, kits, and methods for medical delivery of multiple fluid (e.g., liquid) agents (e.g., at least two) to a patient (e.g., human or animal) from pre-filled (and, in some embodiments, field-assembled or assembled at the point-of-use) modules (also referred to herein as a component, bottle, or vial). In some embodiments, at least two fluid agents are sealed in separate modules and maintained separate from each other until a time when it is desirable to mix the fluid agents (e.g., at the time of or prior to use and/or administration to the patient). The fluid agents can be any type of agent to be injected into or otherwise delivered to a patient and capable of producing a therapeutic effect, either alone or in combination with an active ingredient. Accordingly, the fluid agents can include, but are not limited to, separate vaccines, drugs, medicaments, diluents, active ingredients, etc. that are desirable to combine for use and/or common administration to the patient. For example, in some embodiments, the combination of the fluid agents can form a multi-fluid agent, solution, mixture, suspension, etc. Alternatively or additionally, the fluid agents can be separate components that, when combined, form a vaccine, drug, medicament, etc. For example, in some embodiments, the combination of the fluid agents can comprise a single dose of a therapeutic agent (e.g., vaccine, drug, medicament, etc.). In some embodiments, one or more of the fluid agents in each module can be tracked, monitored, checked for compatibility, etc., such as by utilization of electronic data storage devices (not shown) coupled to the various modules or components of the system.

In some embodiments, each module may comprise a blow-fill-seal (BFS) module that has at least one reservoir (also referred to herein as chamber) prefilled with one or more fluid agents using a BFS manufacturing technique. In some embodiments, the BFS module can have multiple reservoirs, and at least some of the reservoirs filled with fluid agents can be sealed from other of the reservoirs, thereby maintaining the fluid agents separate until combination thereof is desired (e.g., a time for use and/or administration to the patient). The BFS module may be constructed, filled, and sealed, according to some embodiments, in a sterile manufacturing environment. BFS modules may, for example, offer a less expensive alternative to typical vials or bottles created via other manufacturing techniques. In some embodiments, BFS modules (e.g., due to the nature of the BFS manufacturing process) may not require separate sterilization (e.g., and may accordingly be compatible with a wider array of fluid agents), may provide enhanced production rates of sterile/aseptic units per hour, and/or may be provided to an end-user for significantly lower per dose/unit costs. In some embodiments, these advantages may come with attendant drawbacks of reduced manufacturing tolerances and other disadvantages of utilizing a “soft” plastic (e.g., having a Shore/Durometer “D” hardness of between 60 and 70). BFS processes may, for example, offer less precise manufacturing tolerances in the range of five hundredths of an inch (0.05-in; 1.27 mm) to fifteen hundredths of an inch (0.15-in; 3.81 mm)—for linear dimensions, e.g., in accordance with the standard ISO 2768-1 “General tolerances for linear and angular dimensions without individual tolerance indications” published by the International Organization for Standardization (ISO) of Geneva, Switzerland (Nov. 15, 1989), which is incorporated herein by reference.

In some embodiments, a connector (also referred to herein as a manifold, coupling, or fluid sub-assembly) can be coupled to a BFS module to breach a fluid seal thereof, thereby allowing fluidic access to the reservoir (e.g., fluid agent can be added to and/or removed from the reservoir) of the BFS module. In some embodiments, the BFS module can have a neck that has a laterally-protruding portion that interfaces with a mating feature in a recess (also referred to herein as bore, port, chamber, or opening) of the connector when the neck is inserted therein, which can facilitate coupling of the BFS module to the connector. In some embodiments, a pair of connectors are releasably coupled together in order to allow the fluid agent from one BFS module to be combined with the fluid agent from another BFS module prior to use and/or administration. In some embodiments, the combination (e.g., mixing) can occur within the reservoir of one of the BFS modules. For example, actuation (e.g., lateral or longitudinal compression) of a first BFS module dispenses a first fluid agent from the reservoir of the first BFS module via the coupled connectors into the reservoir of the second BFS module, thereby combining the first fluid agent with a second fluid agent in the second BFS module.

In some embodiments, the combined fluid agents can be administered to a patient via an administration assembly coupled to one of the connectors. In some embodiments, the first connector with first BFS module (e.g., partially or fully emptied, exhausted, or spent) can be decoupled from the second connector, for example, to allow the administration assembly to be releasably coupled to the second connector. With the administration assembly and second connector coupled together, the reservoir of the second BFS module can be fluidically connected to an outlet of the administration assembly. Actuation (e.g., lateral or longitudinal compression) of the second BFS module dispenses the combined fluid agents from the second reservoir along the second flow path and through the outlet of the administration assembly. For example, embodiments of the disclosed subject matter can deliver the combination of fluid agents from the BFS module as a single dose of a therapeutic agent (e.g., vaccine, drug, medicament, etc.).

In some embodiments, the administration assembly comprises a needle or cannula constructed for subcutaneous, intramuscular, intradermal, or intravenous injection of the combined fluid agents into the patient. Alternatively, in some embodiments, the administration assembly comprises a nozzle. For example, the nozzle can be a spray nozzle that facilitates dispersion of the combined liquid agents into a spray, which configuration may be useful in the administration of the combined fluid agents into a body cavity or orifice (e.g., nasal passage, ear canal, etc.). In another example, the nozzle can be a droplet nozzle that facilitates formation of droplets of the combined fluid agents, which configuration may be useful in the administration of the combined fluid agents to the eyes, for topical application, etc.

In some embodiments, the administration assembly, the first connector, and/or the second connector may be configured to be coupled and/or assembled to the BFS modules on-site and/or in the field. Alternatively or additionally, in some embodiments, a connector may be coupled and/or assembled to a BFS module in a manufacturing facility and provided to users as a single, pre-assembled fluid sub-assembly (e.g., with a neck of the BFS module inserted into a recess of the connector, but without breach of the sealed port of the BFS module). In some embodiments, the pre-filled multi-fluid medical delivery assembly may, for example, be capable of delivering combined fluid agents in a controlled manner and without requiring specialized skill in assembling and/or administering delivery of such agents.

### II. Pre-filled Multi-Fluid Medical Delivery Assemblies

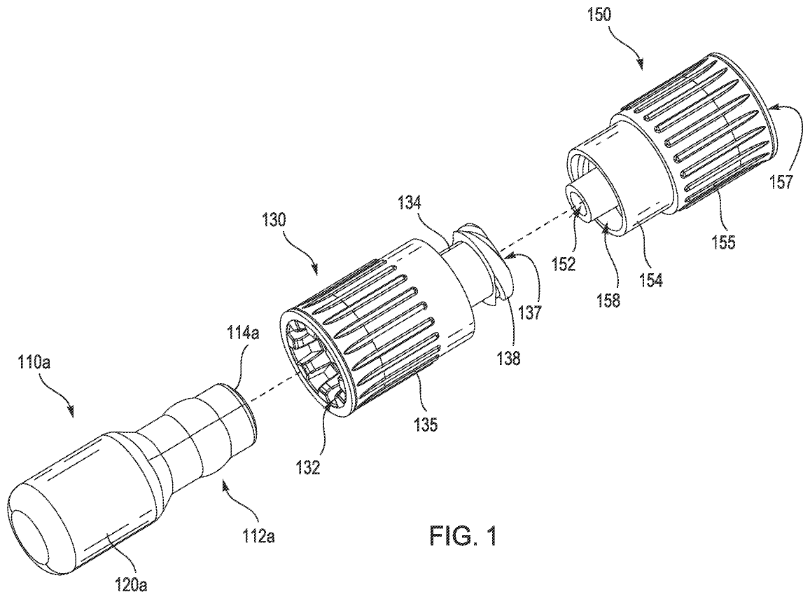

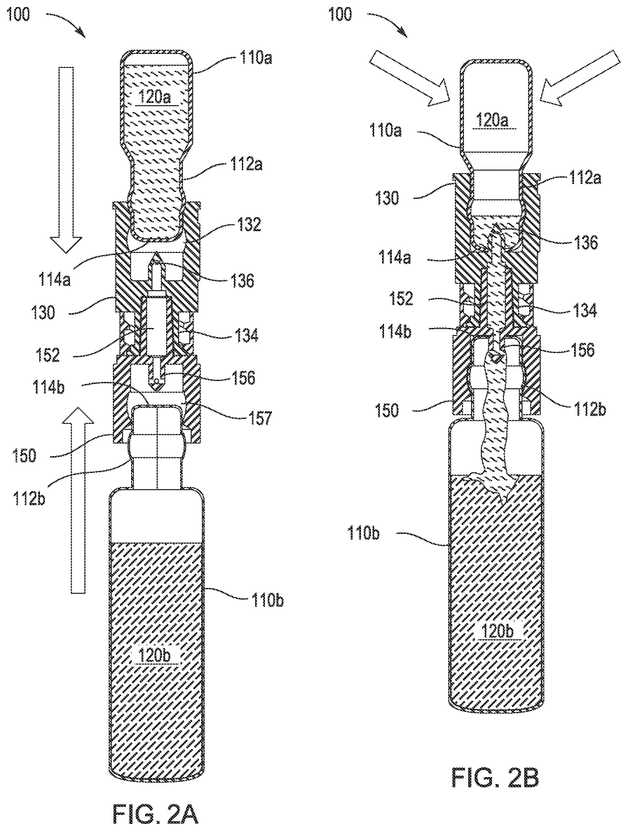

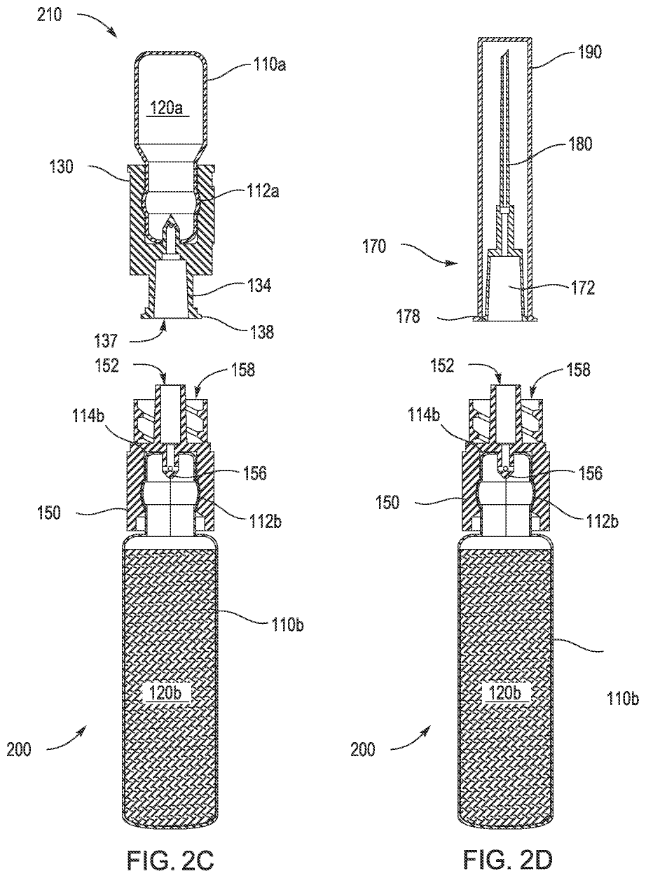

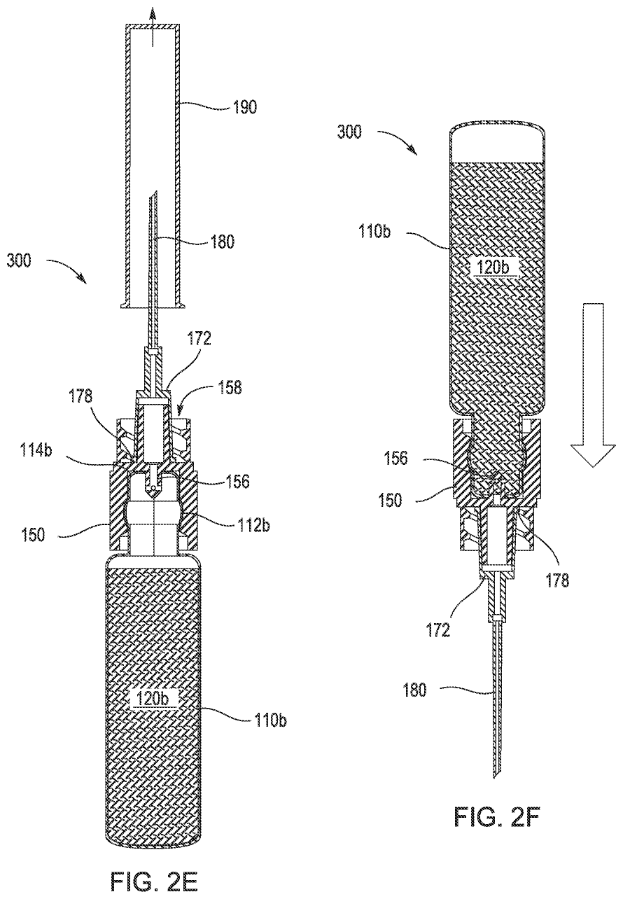

Referring initially to FIGS. 1-2C, various views of a pre-filled multi-fluid assembly 100 according to some embodiments are shown. In some embodiments, the pre-filled multi-fluid assembly 100 may comprise various inter-connected and/or modular components, for example, a first BFS module 110a, a second BFS module 110b, a first connector 130, and a second connector 150. In some embodiments, the first BFS module 110a, the second BFS module 110b, or both may be constructed (e.g., formed) via a BFS process and may comprise a “soft” plastic (e.g., having a Shore/Durometer “00” hardness of between 60 and 70 and/or a Shore/Durometer “A” hardness between 20 and 50) that is not functionally susceptible to the formation and/or utilization of threaded connection features (as are possible to form on different, harder plastics).

In some embodiments, the first BFS module 110a can comprise and/or define a first neck portion 112a that extends longitudinally and is sealed via a first fluid seal 114a at a terminal end thereof, and/or the second BFS module 110b can comprise and/or define a second neck portion 112b that extends longitudinally and is sealed via a second fluid seal 114b at a terminal end thereof. In some embodiments, each BFS module can have at least one fluid reservoir. For example, the first BFS module 110a can comprise and/or define a first fluid reservoir, and/or the second BFS module 110b can comprise and/or define a second fluid reservoir. In some embodiments, each neck portion 112a, 112b can be in fluid communication and axially aligned with a respective fluid reservoir 120a, 120b. In some embodiments, the first and second neck portions 112a, 112b can be substantially cylindrical and/or can extend longitudinally at an end of the respective BFS module 110a, 110b. In some embodiments, the first neck portion 112a, the second neck portion 112b, or both can have a surface feature, such as a laterally-protruding portion, for retaining the neck portion 112a, 112b within a recess of the respective connector. For example, the surface feature of the neck portion can have comprise a toroidal or doughnut shaped protrusion.

In some embodiments, the fluid reservoirs 120a, 120b may be filled (fully or partially) with a fluid or other agent (not separately labeled) to be delivered, e.g., to a patient (not shown). According to some embodiments, each fluid reservoir 120a, 120b may be filled with a different fluid agent (e.g., having different composition, phase, volume, or any combination of the foregoing), such as a different liquid or gas. Such different fluids agents can be combined, introduced, and/or mixed via the pre-filled multi-fluid assembly 100 (e.g., via the first connector 130 and the second connector 150) to define a combined fluid agent for use or administration to a patient. For example, the first fluid reservoir 120a can contain a first fluid agent, and the second fluid reservoir 120b can contain a second fluid agent. In some embodiments, the second fluid reservoir 120b may house or contain a second volume (e.g., two to three milliliters (2.0 to 3.0-ml)) of, for example, a vaccine or second agent (e.g., Pralidoxime). Alternatively or additionally, in some embodiments, the first fluid reservoir 120a may house or contain a first volume (e.g., seven tenths milliliters (0.7-ml)) of, for example, an adjuvant (and/or carrier fluid, catalyst, diluent, etc.) or first agent (e.g., Atropine).

According to some embodiments, the first fluid reservoir 120a can have a first capacity (e.g., a volume for fluid agents), and/or the second fluid reservoir 120b can have a second capacity (e.g., a volume for fluid agents). In some embodiments, the second capacity can be greater than the first capacity, and/or the volume of the second fluid agent contained within the second fluid reservoir 120b can be greater than the volume of the first fluid agent contained within the first fluid reservoir 120a. For example, the first fluid reservoir 120a may be sized to house approximately seven tenths of a milliliter (0.7-ml) of a first fluid agent and/or the second fluid reservoir 120b may be sized to house approximately three milliliters (3.0-ml) of a second fluid agent.

According to some embodiments, any or all of the fluid agents may be injected into the BFS modules 110a, 110b (e.g., into respective fluid reservoirs 120a, 120b thereof) in a sterile environment during manufacture via a BFS process and sealed within the BFS modules 110a, 110b via fluid seals 114a, 114b. In some embodiments, the fluid seals 114a, 114b may comprise portions of the molded BFS modules 110a, 110b, for example, that are configured to be breached (e.g., pierced) to expel the respective fluids. In some embodiments, the first fluid seal 114a, second fluid seal 114b, or both can be formed during a BFS manufacturing process. Alternatively or additionally, in some embodiments, fluid seal 114a, fluid seal 114b, or both may comprise foil, wax, paper, plastic, and/or other thin, pierceable objects or layers coupled to (or formed as part of) the BFS modules 110a, 110b. In some embodiments, one or both of fluid seals 114a, 114b can provide a flat or planar piercing surface and/or can be oriented normal to an axis of the BFS modules 110a, 110b (and/or the pre-filled multi-fluid assembly 100). In some embodiments, the seals 114a, 114b can be breached, for example, by puncturing, piercing, rupturing, penetrating, or otherwise breaking the seal, in order to access and/or dispense contents of the fluid reservoirs 120a, 120b.

According to some embodiments, the pre-filled multi-fluid assembly 100 may comprise a first connector 130 that comprises and/or defines a first piercing element 136 and/or a second connector 150 that comprises and/or defines a second piercing element 156. The first piercing element 136 can be disposed within and/or formed as part of a first recess 132 (e.g., bore, port, chamber, etc.), and/or the second piercing element 156 can be disposed within and/or formed as part of a second recess 157 (e.g., bore, port, chamber, etc.). In some embodiments, first piercing element 136, second piercing element 156, or both define and/or comprise an “X” and/or cross-shaped configuration (e.g., with one cross-member protruding higher than the other) and/or may be integral to the respective connector 130, 150. Alternatively, in some embodiments, first piercing element 136, second piercing element 156, or both may be constructed of a material (e.g., metal) than that of the connectors 130, 150, for example, a thermoplastic polymer or other relatively hard plastic (e.g., greater than 80 on the Rockwell “M” scale; e.g., Rockwell M 85; and/or greater than 110 on the Rockwell “M” scale; e.g., Rockwell R 115), such as, but not limited to, polypropylene, polybenzimidazole, acrylonitrile butadiene styrene (ABS), polystyrene, polyvinyl chloride, polycarbonate, or the like. Alternatively or additionally, in some embodiments, first piercing element 136, second piercing element 156, or both may be coupled to the respective connector 130, 150.

In some embodiments, the first BFS vial 110a may be coupled with and/or mated to the first connector 130, for example, by longitudinally inserting the first neck 112a into the first recess 132, such that first piercing element 136 approaches (as shown in FIG. 2A) and then breaches (as shown in FIG. 2B) the fluid seal 114a. In some embodiments, the second BFS module 110b may be coupled with and/or mated to the second connector 150, for example, by longitudinally inserting the second neck 112b into the second recess 157, such that second piercing element 156 approaches (as shown in FIG. 2A) and then breaches (as shown in FIG. 2B) the fluid seal 114b. In some embodiments, prior to or after coupling the BFS vials to the respective connectors, the first connector 130 can be coupled with and/or mated to the second connector 150. For example, the first connector 130 can comprise and/or define a longitudinally-extending member 134 forming an outlet port 137 and a laterally-extending engagement member 138 (e.g., flange), and/or the second connector 150 can comprise and/or define an inlet/outlet (I/O) port 152 surrounded by an annular hub 154 with threads 158. The member 134 of the first connector 130 can be constructed to engage with the threads 158 of the second connector 150 when the I/O port 152 is inserted into outlet port 137 and the connectors are rotated with respect to each other about a longitudinal axis. Alternatively or additionally, the second connector 150 may comprise threads that correspond and cooperate with threads of the first connector 130 such that they may be rotationally and/or removably coupled.

In some embodiments, one or both of the connectors can comprise and/or define a knurled surface (e.g., surface 135 for the first connector or surface 155 for the second connector). Each connector 130, 150 may also comprise and/or define an internal fluid passage (not separately labeled), for example, that permits fluid to flow between the first recess 132 and the second recess 157 via the connected ports 137, 152. In some embodiments, and as depicted in FIGS. 1-2A, the components 110a, 110b, 130, and 150 of the medical fluid assembly 100 may be assembled in a longitudinal (e.g., axial) configuration and longitudinal force may be applied to engage each BFS vial 110a, 110b with the respective connector 130, 150. The axial force may cause, in some embodiments, the piercing elements 136, 156 to pierce the seals 114a, 114b of the respective BFS modules 110a, 110b, thereby placing the first and second fluids in communication via the internal fluid passages of the connectors 130, 150.

According to some embodiments, the connectors 130, 150 can be coupled (e.g., releasably or removably) with the BFS modules 110a, 110b via application of a longitudinal mating force. In some embodiments, one or both of the connectors 130, 150 can comprise and/or define mating features, for example, one or more interior grooves, slots, recesses, indents, tracks, apertures, etc., sized and/or positioned to selectively couple to and/or index with portions of necks 112a, 112b of the corresponding BFS vial 110a, 110b. In some embodiments, the necks of the BFS vial and the mating features of the connectors may be cooperatively shaped to permit the BFS vial to be snapped or “clicked” into the connector. For example, in some embodiments, the BFS module 110a can be urged into the first recess 132 (e.g., an open longitudinal end of the connector 130), for example, such that cooperatively-shaped interior chambers and/or grooves (e.g., scalloped sidewalls, not shown) accept the first neck portions 112a (e.g., with cooperating surface features, such as a toroidal or doughnut shaped protrusion), thereby removably coupling the first BFS module 110a to the first connector 130. Alternatively or additionally, the BFS module 110b can be urged into the second recess 157 (e.g., an open longitudinal end of the connector 150), for example, such that cooperatively-shaped interior chambers and/or grooves accept the second neck portions 112b (e.g., with cooperating surface features, such as a toroidal or doughnut shaped protrusion), thereby removably coupling the second BFS module 110b to the second connector 150. In some embodiments, the interior chambers and/or grooves (and/or other interior features) and/or the neck portions 112a, 112b may be shaped such that uncoupling of the BFS modules 110 from the respective connectors 130, 150 is mechanically prohibited, or at least resisted. According to some embodiments, insertion of the neck portions 112a, 112b into the connectors 130, 150 and/or otherwise mating thereof may cause the reservoirs 120a, 120b to remain outside of and/or axially adjacent to the connectors 130, 150 (e.g., exposed from the connectors).

According to some embodiments, once the BFS modules 110a, 110b are fully engaged with the respective connectors 130, 150 such that the seals thereof have been pierced by the respective piercing elements 136, 156 (e.g., as shown in FIG. 2B), the contents in the reservoir 120a of the first BFS module 110a may be dispensed, for example, by application of a radial, squeezing, and/or other force to the exposed portion of the first BFS module 110a (e.g., to the exterior wall surrounding the reservoir 120a). The fluid agent from the first BFS module 110a can be dispensed into the second BFS module 110b, for example, via the internal fluid passage of the connectors 130, 150, as shown in FIG. 2B.

In some embodiments, once sufficient dispensing from the first BFS vial 110a has been achieved (e.g., once the reservoir 120a has been emptied), the first connector 130 may be disengaged from the second connector 150, as shown in FIG. 2C, for example, providing a first fluid subassembly 210 comprised of the first connector 130 coupled to the first BFS module 110a and a second fluid subassembly 200 comprised of the second connector 150 coupled to the second BFS module 110b. In some embodiments, one or both of the components of the first fluid subassembly 210 (e.g., the first connector 130 and the first BFS module 110a) may be discarded.

According to some embodiments, removal of the first connector 130 may expose threads 158 of the second connector 150 that may be utilized to couple to and/or retain an administration assembly 170, as shown in FIG. 2D, to the second fluid subassembly 200, thereby forming a medical delivery assembly 300, as shown in FIG. 2E. In some embodiments, the administration assembly 170 may comprise and/or define a hub 172 that retains an administration member 180. In some embodiments, the hub 172 can comprise and/or define a laterally-extending flange 178 or threads that that couples to (e.g., screwed onto) or engages with the threads 158 of the second connector 150. Alternatively or additionally, the hub 172 may comprise threads that correspond and cooperate with threads of the second connector 150 such that they may be rotationally and/or removably coupled. In some embodiments, an outlet end and/or a distal portion of the administration member 180 may be housed, shrouded, and/or covered by a cap 190. According to some embodiments, the cap 190 may be configured to house the administration member 180 and to be removably coupled to the hub 172 (e.g., by fitting over an external portion thereof).

According to some embodiments, the connector 150 and hub 172 combination may be utilized to couple and/or mate the administration member 180 with the BFS module 110b to provide a mechanism by which the administration member 180 may be coupled to the soft plastic BFS module 110b in a reliable manner. Due to the nature of the BFS plastic and/or process and/or the small form-factor of the BFS module 110b, for example, providing external threads directly on the BFS module 110b would not be a viable option, as it may result in an imprecise, unreliable, and/or non-water tight coupling (e.g., the threads would be deformable even if they could be properly manufactured to within the desired tolerances, which itself is not a likely result) between the BFS module 110b and the hub 172. Applicant has realized, for example, that the “soft” plastics required for BFS process are not susceptible to machining due to heat deformation of machined features during formation attempts as well as deformation due to mechanical stress during utilization.

In some embodiments, the administration member 180 may comprise a needle for at least one of subcutaneous, intramuscular, intradermal, and intravenous injection of the fluid agent into the patient. For example, the needle can have a length of 0.5 mm to 4 mm, inclusive, or in a range of 4 mm to 15 mm, inclusive, or in a range of 15 mm to 30 mm, inclusive, depending on the desired manner of injection. For ease of explanation and description, the figures and the description herein generally refer to the administration member as a needle. However, it should be noted that, in other embodiments, the administration member 180 may include a nozzle (not shown) configured to control administration of the fluid agent to the patient. The nozzle may include a spray nozzle, for example, configured to facilitate dispersion of the fluid agent into a spray. Accordingly, a hub 172 fitted with a spray nozzle may be particularly useful in the administration of a fluid agent into the nasal passage, for example, or other parts of the body that benefit from a spray application (e.g., ear canal, other orifices). In other embodiments, the nozzle may be configured to facilitate formation of droplets of the fluid agent. Thus, a hub 172 including a droplet nozzle may be useful in the administration of a fluid agent by way of droplets, such as administration to the eyes, topical administration, and the like.

In some embodiments, and as depicted in FIG. 2E, the cap 190 may be removed to expose the outlet end of the administration member 180. As depicted in FIG. 2F, the pre-filled multi-fluid medical delivery assembly 100 may then be positioned for use and/or administration (e.g., injection; e.g., inverted vertically, as shown for purposes of example only). The combined multi-fluid agent housed in the second reservoir 120b may then, for example, be injected into a target such as by applying pressure to the second BFS module 110b (e.g., by laterally compressing or squeezing the second BFS module 110b proximal to the second reservoir 120b). As is generally understood, the fluid (e.g., combined fluid) or drug agent may include any type of agent to be injected into a patient (e.g., mammal, either human or non-human, or any other animal) and capable of producing an effect (alone, or in combination with an active ingredient). Accordingly, the agent may include, but is not limited to, a vaccine, a drug, a therapeutic agent, a medicament, a diluent, and/or the like. According to some embodiment, either or both of the fluid agent and the active ingredient (i.e., the drug agent and/or components thereof) may be tracked, monitored, checked for compatibility with each other, etc., such as by utilization of electronic data storage devices (not shown) coupled to the various modules or components of the pre-filled multi-fluid medical fluid assembly 100, such as the BFS modules 110a, 110b and/or the connectors 130, 150.

In some embodiments, the pre-filled medical delivery assembly or system may comprise various inter-connected and/or modular components. For example, the pre-filled multi-fluid medical assemblies may include a modular design consisting of separately constructed components 110a, 110b, 130, 150, and 170, cooperatively arranged and coupled to one another. In some embodiments, some or all of the various components can be provided in an unassembled state as a kit, for example, for assembly in the field and/or at a time just prior to use or administration. In some embodiments, the medical fluid assembly 100, the medical delivery assembly 300, or the kit for forming either or both assemblies 100, 300, can include additional components beyond those specifically illustrated in FIGS. 1-2F. In some embodiments, the components of the kit, e.g., the BFS modules 110a, 110b, the connectors 130, 150, and the administration assembly 170 can be manufactured, packaged, shipped, stored, and/or provided as separate components. In such a manner, the connectors and/or the administration assembly may not need to be stored or shipped in accordance with often restrictive requirements imposed on medicaments and may accordingly reduce the amount of space required for such specialized storage and/or shipping. The connectors and/or the administration assembly may also or alternatively be manufactured, stored, and/or shipped in advance (e.g., at a first time) while the BFS modules 110a, 110b that are pre-filled with the fluid agents may be manufactured, stored, and/or shipped at a later time (e.g., a second time). In some embodiments, the delay between the first time and the second time may be lengthy without causing detrimental effects, as the connectors and/or the administration assembly may be stored, in some embodiments, indefinitely. In such a manner, units of the connectors and/or the administration assembly may be provided to be on-hand in advance of the availability and/or arrival of the BFS modules 110a, 110b, reducing supply chain constraints in the case of proactive mixing assembly procurement. Alternatively or additionally, at least some components of the assembly may be reusable with other BFS modules, for example, connector 130 and/or connector 150. Thus, unassembled components of pre-filled medical delivery system kits according to embodiments of the disclosed subject matter need not be provided together in the same package or at the same time.

In some embodiments, fewer or more components 110-300 and/or various configurations of the depicted components 110-300 may be included in the pre-filled multi-fluid medical fluid assembly, the multi-fluid medical delivery assembly, and/or sub-assemblies thereof without deviating from the scope of embodiments described herein. In some embodiments, the components 110-300 may be similar in configuration and/or functionality to similarly named and/or numbered components as described herein.

### III. Pre-Filled Multi-Fluid Medical Delivery Methods

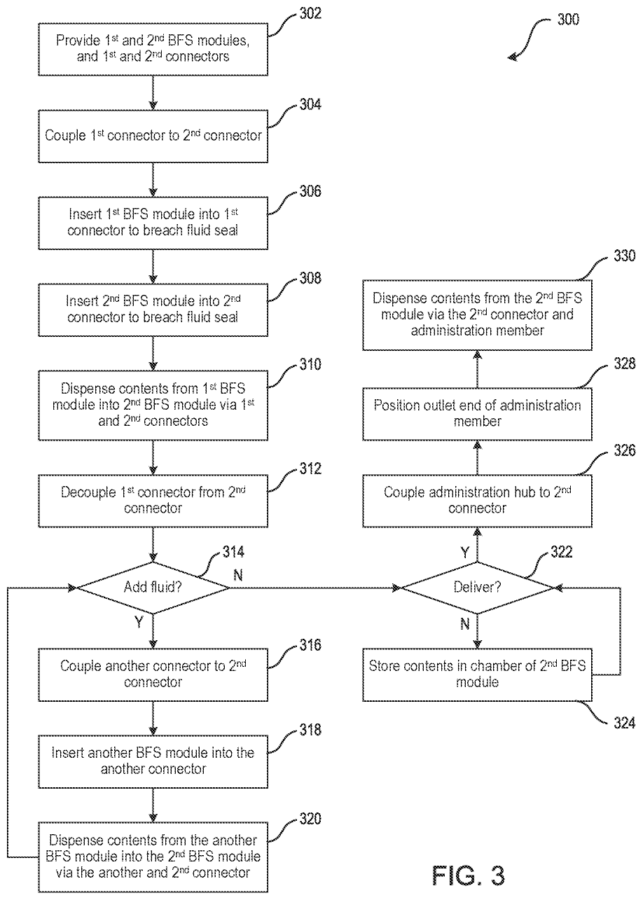

FIG. 3 illustrates an exemplary method 350 for use and/or administration of combined fluid agents using a pre-filled medical delivery assembly. The method 350 can initiate at process block 302, where first and second BFS modules (e.g., BFS vial, bottle, or container), each having at least one reservoir storing a respective fluid agent therein, are provided. For example, the first and second BFS modules can be similar to any of the modules described above with the respect to FIGS. 1-2F. Alternatively or additionally, the first BFS module, the second BFS module, or both can have a multi-chamber configuration, for example, as described in International Publication No. WO 2021/207040, published Oct. 14, 2021 and titled “Systems and Methods for Pre-filled Medical Delivery,” which is incorporated by reference herein in its entirety. In some embodiments, the provision of process block 302 can include manufacturing the first BFS module with first fluid agent sealed therein and/or manufacturing the second BFS module with second fluid agent sealed therein, for example, using a BFS manufacturing technique.

The method 350 can proceed to process block 304, where first and second connectors can be coupled together. For example, the first and second connectors can be similar to the connectors described above with respect to FIGS. 1-2F. The coupled first and second connectors can provide a fluid conduit between corresponding recesses of the connectors. The first and second connectors can be releasably coupled together. For example, the releasable coupling can comprise a Luer lock connection (e.g., defined by ISO Standard 80369-7:2016, “Small-bore connectors for liquids and gases in healthcare applications—Part 7: Connectors for intravascular or hypodermic applications,” incorporated by reference herein), where one of the first and second connectors has a threaded hub or collar and the other of the first and second connectors has a laterally-extending member or flange (e.g., one or more Luer-style thread protrusions) for engaging with the threads of the hub. Alternatively or additionally, the releasable coupling can employ other types of fluid connections, such as, but not limited to a Luer slip connection (e.g., a tapered male fitting inserted into a tapered female fitting), twist-to-connect couplings, twist-claw couplings, etc.

The method 350 can proceed to process block 306, where the first BFS module is inserted into the first connector, and to process block 308, where the second BFS module is inserted into the second connector. For example, a neck portion of the first BFS module can be inserted along a longitudinal direction into a first recess of the first connector, and/or a neck portion of the second BFS module can be inserted along a longitudinal direction into a second recess of the second connector. In some embodiments, the recess of each connector can include a piercing element configured to breach a fluid seal of a BFS module. For example, when the neck portion of a BFS module is fully inserted into the recess (e.g., such that further progress into the recess is retarded or prevented) of the corresponding connector, the piercing element of the corresponding connector breaches (e.g., pierces, breaks, punctures, ruptures and/or extends through) the seal of the BFS module, thereby allowing fluid communication between the reservoir within the BFS module and the connector, and thus between the reservoirs of the first and second BFS modules while the first and second connectors are coupled together.

The method 350 can proceed to process block 310, where some or all of the contents (e.g., a first fluid agent) in the reservoir of the first BFS module are dispensed into the reservoir of the second BFS module via the coupled first and second connectors. For example, a user can actuate the first BFS module by compressing (e.g., by pressing laterally, by pressing longitudinally, or both) the reservoir of the first BFS module. The resulting pressure forces the first fluid agent from the reservoir through the breached port of the first BFS module, through the first and second connectors, through the breached port of the second BFS module, and into the reservoir of the second BFS module, where it can combine (e.g., mix) with the second fluid agent therein. In some embodiments, a volume of the reservoir of the second BFS module can be sufficient to accommodate the volumes of both the first and second fluid agents. For example, the volume of the reservoir of the second BFS module can be greater (e.g., at least two times greater) than the volume of the reservoir of the first BFS module. In some embodiments, the second BFS module (e.g., with both connectors and the first BFS module coupled thereto, or with only the second connector coupled thereto) can be shaken after the first fluid agent is dispensed into the second BFS module, for example to ensure sufficient emptying of reservoir of the first BFS module and/or adequate mixing of the fluid agents in the reservoir of the second BFS module.

The method 350 can proceed to process block 312, where the first connector is decoupled (e.g., disconnected, removed, unscrewed, etc.) from the second connector. In some embodiments, the first BFS module can remain inserted into the first connector and the second BFS module can remain inserted into the second connector as the first connector is decoupled from the second connector. Alternatively, in some embodiments, the first BFS module can be removed from the first connector prior to decoupling the first connector from the second connector.

The method 350 can proceed to decision block 314, where it is determined if additional fluid agents should be combined with the first and second fluid agents in the second BFS module. If additional fluid agents are desired, the method 350 can proceed to process block 316, where another connector (e.g., a third connector similar to the first connector) is coupled to the second connector, for example, in a manner similar to process block 304. The method 350 can proceed to process block 318, where another BFS module (e.g., a third BFS module) is inserted into the another connector, for example, in a manner similar to process block 306. The method 350 can then proceed to process block 320, where some or all of the contents (e.g., a third fluid agent) in the reservoir of the another BFS module are dispensed into the reservoir of the second BFS module via the coupled another and second connectors, for example, in a manner similar to process block 310. The method 350 can then return to decision block 314 for consideration if additional fluid agents (e.g., fourth fluid agent, fifth fluid agent, etc.) are desired.

If no additional fluid agents are desired at decision block 314, the method 350 can proceed to decision block 322, where it is determined if dispensing of the combined fluid agents is desired. If dispensing is not yet desired, the method 350 can proceed to process block 324, where the second BFS module, with second connector coupled thereto and with combined fluid agents contained in the reservoir thereof, can be stored for later use. In some embodiments, after removal of the first connector, a fluid-tight cap (e.g., seal) can be releasably coupled to the second connector, for example, to prevent contamination and/or premature dispensing of the contents in the reservoir of the second BFS module. In some embodiments, the storing of process block 324 can include providing appropriate environment conditions (e.g., a temperature at or below room temperature (e.g., 20-22° C.)) for the second BFS module to maintain viability of the fluid agents contained therein.

If dispensing is desired, the method 350 can proceed from decision block 322 to process block 326, where an administration assembly is coupled to the second connector. For example, the administration assembly can be similar to the assembly described above with respect to FIGS. 2D-2F. The coupled administration assembly and second connector can provide a fluid conduit from the recess of the second connector to an output end of an administration member of the administration assembly. In some embodiments, the administration assembly and the second connector can be releasably coupled together. For example, the releasable coupling can comprise a Luer lock connection, a Luer slip connection, twist-to-connect couplings, twist-claw couplings, or any other type of fluid connection. Alternatively or additionally, the coupling between the administration assembly and the second connector can be non-releasable (e.g., permanent), for example, employing an adhesive.

In some embodiments, after process block 326, the assembly may be ready for use and/or administration. The method 350 can proceed to process block 328, where the outlet end of the administration assembly is positioned for subsequent dispensing of the combined fluid agents contained in the second BFS module. For example, an outlet end of the administration member can be inserted into or positioned with respect to a patient. If the administration assembly includes a protective cap, it can be removed prior to positioning the outlet end of the administration member. In some embodiments, the administration member can comprise a needle, and the inserting can comprise inserting an outlet end of the needle into the patient, for example, to effect a subcutaneous, intramuscular, intradermal, and intravenous injection of the combined liquid agents. Alternatively, in some embodiments, the administration member can comprise a nozzle, and the inserting can comprise inserting an outlet end of the nozzle into an orifice or cavity of the patient (e.g., oral, nasal, ear canal, etc.) or disposing adjacent to an exposed surface of the patient (e.g., for topical application, eyes, etc.)

The method 350 can then proceed to process block 330, where the combined fluid agents contained in the reservoir of the second BFS module can be dispensed, for example, for use or administration to a patient via the outlet end of the administration member. For example, a user can actuate the second BFS module by compressing (e.g., by pressing laterally, by pressing longitudinally, or both) the reservoir of the second BFS module. The resulting pressure forces the contents in the mixing chamber out of the manifold through the administration member. The combined fluid agents from the reservoir through the breached port of the second BFS module, through the second connector, through the administration member, and out of the outlet end of the administration member, so as to dispense the combined fluid agents. In some embodiments, the dispensing of combined fluid agents of process block 330 is effective to a deliver a single dose of a therapeutic agent to the patient, for example, having a volume of 3 ml or less. In some embodiments, the administration member can be removed from the patient after dispensing the fluid agents.

In some embodiments, the method can also include disposing of some or all of the components of the pre-filled medical delivery assembly. For example, each BFS module can be constructed for single use and may be incapable of (or at least undesirable for) reuse once the seals are breached and the fluid agents are dispensed therefrom. In some embodiments, the first connector, the second connector, and/or the administration assembly can also be constructed for single use and may be discarded after the administration. Alternatively or additionally, in some embodiments, the first connector, the second connector, and/or the administration assembly can be retained for reuse, with or without cleaning and/or sterilization.

Although some of blocks 302-330 of method 350 have been described as being performed once, in some embodiments, multiple repetitions of a particular process block may be employed before proceeding to the next decision block or process block. In addition, although blocks 302-330 of method 350 have been separately illustrated and described, in some embodiments, process blocks may be combined and performed together (simultaneously or sequentially).

Moreover, although FIG. 3 illustrates a particular order for blocks 302-330, embodiments of the disclosed subject matter are not limited thereto. Indeed, in certain embodiments, the blocks may occur in a different order than illustrated or simultaneously with other blocks. For example, in some embodiments, the first BFS module can be inserted into the first connector prior to coupling the first connector to the second connector, the second BFS module can be inserted into the second connector prior to coupling the first connector to the second connector, or both. Alternatively or additionally, in some embodiments, the first BFS module can be partially inserted into the first connector (e.g., without breaching the seal of the first BFS module) prior to or after coupling the first connector to the second connector, the second BFS module can be partially inserted into the second connector (e.g., without breaching the seal of the second BFS module) prior to or after coupling the first connector to the second connector, or both. In such embodiments, after the first and second connectors are coupled together, the BFS module can then be further inserted into the respective connector to breach the respective seal.

### IV. Additional Examples of the Disclosed Technology

In view of the above described implementations of the disclosed subject matter, this application discloses the additional examples in the clauses enumerated below. It should be noted that one feature of a clause in isolation, or more than one feature of the clause taken in combination, and, optionally, in combination with one or more features of one or more further clauses are further examples also falling within the disclosure of this application.

Clause 1. A pre-filled medical delivery assembly, comprising:
a first blow-fill-seal (BFS) module defining a first fluid reservoir;
a first connector comprising a first chamber into which the first BFS module may be seated, a first piercing element coupled to align with a sealed terminus of the first BFS module in the case that the first BFS module is inserted into the first chamber, and an internal fluid passage disposed through the first piercing element;
a second blow-fill-seal (BFS) module defining a second fluid reservoir; and
a second connector comprising a second chamber into which the second BFS module may be seated, a second piercing element coupled to align with a sealed terminus of the second BFS module in the case that the second BFS module is inserted into the second chamber, and an internal fluid passage disposed through the second piercing element;
wherein the first and second connectors are axially coupled together and engaged with the respective BFS modules thereby causing the respective piercing elements to pierce the BFS modules and thereby placing the first and second fluid reservoirs in fluid communication with each other.

Clause 2. The assembly of any clause or example herein, in particular clause 1, wherein the first fluid reservoir is less than half of the volume of the second fluid reservoir.

Clause 3. A pre-filled medical delivery assembly comprising:
(i) a first blow-fill-seal (BFS) module having a first reservoir having a first fluid agent therein and a first neck with a first seal at an end thereof, the first neck being in fluid communication with the first reservoir;
(ii) a second BFS module having a second reservoir having a second fluid agent therein and a second neck with a second seal at an end thereof, the second neck being in fluid communication with the second reservoir;
(iii) a first connector having a first recess constructed to receive at least part of the first neck of the first BFS module and an outlet port in fluid communication with the first recess, the first recess having a first piercing element therein; and
(iv) a second connector having a second recess constructed to receive at least part of the second neck of the second BFS module and an inlet/outlet (I/O) port in fluid communication with the second recess, the second recess having a second piercing element therein,
wherein the first and second connectors are constructed to be coupled together via the outlet port and the I/O port, so as to provide a fluid path between the first and second reservoirs.

Clause 4. The assembly of any clause or example herein, in particular, any one of clauses 1-3, wherein (a) an end of the first connector having the outlet port has threads for coupling to the second connector, (b) an end of the second connector having the I/O port has threads for coupling to the first connector, or both (a) and (b).

Clause 5. The assembly of any clause or example herein, in particular, any one of clauses 1-4, wherein:
the first connector is disposed between the first BFS module and the second connector with respect to a longitudinal direction of the first BFS module;
the second connector is disposed between the second BFS module and the first connector with respect to a longitudinal direction of the second BFS module;
the first and second connectors are disposed between the first and second BFS modules with respect to a longitudinal direction of either the first or second BFS module; or any combination of the foregoing.

Clause 6. The assembly of any clause or example herein, in particular, any one of clauses 1-5, wherein a first longitudinal direction of the first BFS module extends from the first reservoir toward the first seal, a second longitudinal direction of the second BFS module extends from the second reservoir toward the second seal, and the first and second longitudinal directions are co-axial, substantially co-axial (e.g., within 5°), and/or parallel.

Clause 7. The assembly of any clause or example herein, in particular, clause 6, wherein a central axis of the outlet port, a central axis of the I/O port, or both are co-axial, substantially co-axial (e.g., with 5°), and/or parallel with the first and second longitudinal directions.

Clause 8. The assembly of any clause or example herein, in particular, any one of clauses 1-7, wherein (c) the first piercing element breaches or extends through the first seal, (d) the second piercing element breaches or extends through the second seal, or both (c) and (d).

Clause 9. The assembly of any clause or example herein, in particular, any one of clauses 1-8, wherein (e) a tip of the first piercing element is spaced from the first seal (e.g., the first piercing element does not breach or extend through the first seal), (f) a tip of the second piercing element is spaced from the second seal (e.g., the second piercing element does not breach or extend through the second seal), or both (e) and (f).

Clause 10. The assembly of any clause or example herein, in particular, any one of clauses 1-9, wherein (g) an exterior of the first connector has a knurled surface, (h) an exterior of the second connector has a knurled surface, or both (g) and (h).

Clause 11. The assembly of any clause or example herein, in particular, any one of clauses 1-10, wherein the second connector comprises an annular hub surrounding the I/O port.

Clause 12. The assembly of any clause or example herein, in particular, any one of clauses 1-11, wherein the first recess has a first mating feature constructed to receive a laterally-protruding portion of the first neck, the second recess has a second mating feature constructed to receive a laterally-protruding portion of the second neck, or any combination of the foregoing.

Clause 13. The assembly of any clause or example herein, in particular, any one of clauses 1-11, wherein:
(j) the first recess has a first mating feature, and the first neck has a laterally-protruding portion that is received in the first mating feature;
(k) the second recess has a second mating feature, and the second neck has a laterally-protruding portion that is received in the second mating feature; or both (j) and (k).

Clause 14. The assembly of any clause or example herein, in particular, any one of clauses 12-13, wherein:
(l) with the laterally-protruding portion of the first neck received in the first mating feature, the first piercing element breaches or extends through the first seal;
(m) with the laterally-protruding portion of the second neck received in the second mating feature, the second piercing element breaches or extends through the second seal; or both (l) and (m).

Clause 15. The assembly of any clause or example herein, in particular, any one of clauses 12-14, wherein:
(n) the first mating feature comprises a scalloped portion of a sidewall of the first recess, and the laterally-protruding portion of the first neck has a toroidal shape in a side view;
(o) the second mating feature comprises a scalloped portion of a sidewall of the second recess, and the laterally-protruding portion of the second neck has a toroidal shape in a side view; or both (n) and (o).

Clause 16. The assembly of any clause or example herein, in particular, any one of clauses 1-15, wherein:
(p) a volume of the second reservoir is greater than a volume of the first reservoir;
(q) a volume of the second fluid agent is greater than a volume of the first fluid agent; or both (p) and (q).

Clause 17. The assembly of any clause or example herein, in particular, any one of clauses 1-16, wherein the volume of the second reservoir is at least two times greater than the volume of the first reservoir.

Clause 18. The assembly of any clause or example herein, in particular, any one of clauses 1-17, wherein a volume of the second fluid agent is at least 2 ml (e.g., about 2 ml, about 3 ml, or 2-3 ml), and a volume of the first fluid agent is about 0.7 ml.

Clause 19. The assembly of any clause or example herein, in particular, any one of clauses 1-18, wherein the first fluid agent, the second fluid agent, or both comprise a vaccine, a drug, a medicament, or a component of any of the foregoing.

Clause 20. The assembly of any clause or example herein, in particular, any one of clauses 1-19, wherein the first seal, the second seal, or both the first and second seals comprise a foil, wax, paper, a section of the respective BFS module, or any combination of the foregoing.

Clause 21. The assembly of any clause or example herein, in particular, any one of clauses 1-20, further comprising:
(v) an administration assembly comprising:
   a hub constructed to be coupled to the I/O port of the second connector so as to provide a fluid path from the second reservoir; and
   at least one administration conduit having an outlet at an end thereof longitudinally spaced from the hub and being in fluid communication with the second reservoir so as to receive a combination of the first and second fluid agents therefrom.

Clause 22. The assembly of any clause or example herein, in particular, clause 21, wherein the at least one administration conduit comprises a needle or cannula.

Clause 23. The assembly of any clause or example herein, in particular, clause 22, wherein the needle has a length in a range of 0.5 mm to 4 mm, inclusive, or in a range of 4 mm to 15 mm, inclusive, or in a range of 15 mm to 30 mm, inclusive.

Clause 24. The assembly of any clause or example herein, in particular, clause 21, wherein the outlet of the at least one administration conduit is formed as a nozzle configured to facilitate dispersion of the combination of the first and second fluid agents into a spray, or the outlet of the at least one administration conduit is formed as a nozzle configured to facilitate dispersion of the combination of the first and second fluid agents into one or more droplets.

Clause 25. The assembly of any clause or example herein, in particular, any one of clauses 1-24, wherein the first connector, the second connector, or both the first and second connectors are formed of a material having a hardness greater than that of the first BFS module, the second BFS module, or both the first and second BFS modules.

Clause 26. A kit comprising multiple components to be assembled to form the pre-filled medical delivery assembly of any clause or example herein, in particular, any one of clauses 1-25.

Clause 27. A method comprising:
coupling the first connector to the second connector, coupling the first BFS module to the first connector, and coupling the second BFS module to the second connector of a kit to form the pre-filled medical delivery assembly of any clause or example herein, in particular, any one of clause 1-25; and
combining the first and second fluid agents.

Clause 28. The method of any clause or example herein, in particular, clause 27, further comprising:
decoupling the first connector from the second connector; and
coupling an administration hub to the second connector.

Clause 29. The method of any clause or example herein, in particular, clause 28, further comprising administering the combined fluid agents from the second BFS module to a patient via the administration hub.

Clause 30. The method of any clause or example herein, in particular, any one of clauses 27-29, wherein the first fluid agent, the second fluid agent, or both comprise a vaccine, a drug, a medicament, or a component of any of the foregoing.

Clause 31. The method of any clause or example herein, in particular, any one of clauses 27-30, wherein the seal of the first BFS module, the seal of the second BFS module, or both comprise a foil, wax, paper, a section of the respective BFS module, or any combination of the foregoing.

Clause 32. The method of any clause or example herein, in particular, any one of clauses 27-31, wherein a volume of the second fluid agent is at least two times greater than a volume of the first fluid agent; a volume of the second reservoir is at least two times greater than a volume of the first reservoir; a volume of the first fluid agent is less than a volume of the second fluid agent; and/or a volume of the second fluid agent is 2 ml or less.

Clause 33. The method of any clause or example herein, in particular, any one of clauses 27-32, further comprising, prior to the couplings, filling the first and second fluid agents in the first and second reservoirs, respectively, using a blow-fill-seal technique.

Clause 34. The method of any clause or example herein, in particular, any one of Clauses 27-33, wherein the fluid agents provide a single dose of a therapeutic agent to the patient.

### V. Rules of Interpretation

Any or all of the components disclosed herein can be formed of one or more plastics. In some embodiments, some components (e.g., the BFS vials) can be formed of a relatively soft polymer (e.g., having a Shore/Durometer “D” hardness of between 60 and 70), such as polyethylene (e.g., low density polyethylene (LDPE)), polypropylene, or any other polymer adaptable for use in a BFS manufacturing process. In some embodiments, some components (e.g., the connection assemblies, the administration assemblies, and/or needle caps or covers) can be formed, at least in part, of a relatively hard polymer (e.g., having a hardness greater than 80 on the Rockwell “R” scale), such as, but not limited to, polypropylene, polycarbonate, polybenzimidazole, acrylonitrile butadiene styrene (ABS), polystyrene, polyvinyl chloride, or the like. Other materials are also possible according to one or more contemplated embodiments.

Throughout the description herein and unless otherwise specified, the following terms may include and/or encompass the example meanings provided. These terms and illustrative example meanings are provided to clarify the language selected to describe embodiments both in the specification and in the appended claims, and accordingly, are not intended to be generally limiting. While not generally limiting and while not limiting for all described embodiments, in some embodiments, the terms are specifically limited to the example definitions and/or examples provided. Other terms are defined throughout the present description.

Numerous embodiments are described in this patent application, and are presented for illustrative purposes only. The described embodiments are not, and are not intended to be, limiting in any sense. The presently disclosed invention(s) are widely applicable to numerous embodiments, as is readily apparent from the disclosure. One of ordinary skill in the art will recognize that the disclosed invention(s) may be practiced with various modifications and alterations, such as structural, logical, software, and electrical modifications. Although particular features of the disclosed invention(s) may be described with reference to one or more particular embodiments and/or drawings, it should be understood that such features are not limited to usage in the one or more particular embodiments or drawings with reference to which they are described, unless expressly specified otherwise.

The present disclosure is neither a literal description of all embodiments of the invention nor a listing of features of the invention that must be present in all embodiments.

Neither the Title (set forth at the beginning of the first page of this patent application) nor the Abstract (set forth at the end of this patent application) is to be taken as limiting in any way as the scope of the disclosed invention(s).

While the term “modules” is utilized herein for convenience and ease of illustration, objects represented and/or described as “modules” may comprise various forms, configurations, and/or quantities of components. A BFS module may comprise one or more BFS products that are formed and/or manufactured together or separately, for example, and/or may comprise one or more BFS chambers, bottles, containers, and/or other fluid-retaining objects. The term “module” does not convey any designation of shape or size. In some embodiments, a BFS module may comprise one or more vials. According to some embodiments a BFS module and/or a BFS vial may comprise one or more fluid chambers. In some embodiments, a plurality of BFS modules, components, vials, and/or chambers may be manufactured simultaneously from a single BFS mold. Each respective module and/or chamber may be formed, for example, by different portions of a single BFS mold (e.g., two cooperative halves thereof). In some embodiments, BFS modules, components, vials, and/or chambers may be joined and/or coupled during manufacturing (e.g., via unformed and/or fused connecting parison) and/or after manufacturing/filling.

The term “product” means any machine, manufacture and/or composition of matter as contemplated by 35 U.S.C. § 101, unless expressly specified otherwise.

The terms “an embodiment”, “embodiment”, “embodiments”, “the embodiment”, “the embodiments”, “one or more embodiments”, “some embodiments”, “one embodiment” and the like mean “one or more (but not all) disclosed embodiments”, unless expressly specified otherwise.

A reference to “another embodiment” in describing an embodiment does not imply that the referenced embodiment is mutually exclusive with another embodiment (e.g., an embodiment described before the referenced embodiment), unless expressly specified otherwise.

The terms “a”, “an” and “the” mean “one or more”, unless expressly specified otherwise.

The term “plurality” means “two or more”, unless expressly specified otherwise.

The term “herein” means “in the present application, including anything which may be incorporated by reference”, unless expressly specified otherwise.

The phrase “at least one of”, when such phrase modifies a plurality of things (such as an enumerated list of things) means any combination of one or more of those things, unless expressly specified otherwise. For example, the phrase at least one of a widget, a car and a wheel means either (i) a widget, (ii) a car, (iii) a wheel, (iv) a widget and a car, (v) a widget and a wheel, (vi) a car and a wheel, or (vii) a widget, a car and a wheel.

The phrase “based on” does not mean “based only on”, unless expressly specified otherwise. In other words, the phrase “based on” describes both “based only on” and “based at least on”.

Where a limitation of a first claim would cover one of a feature as well as more than one of a feature (e.g., a limitation such as “at least one widget” covers one widget as well as more than one widget), and where in a second claim that depends on the first claim, the second claim uses a definite article “the” to refer to the limitation (e.g., “the widget”), this does not imply that the first claim covers only one of the feature, and this does not imply that the second claim covers only one of the feature (e.g., “the widget” can cover both one widget and more than one widget).

Each process (whether called a method, algorithm or otherwise) inherently includes one or more steps, and therefore all references to a “step” or “steps” of a process have an inherent antecedent basis in the mere recitation of the term ‘process’ or a like term. Accordingly, any reference in a claim to a ‘step’ or ‘steps’ of a process has sufficient antecedent basis.

When an ordinal number (such as “first”, “second”, “third” and so on) is used as an adjective before a term, that ordinal number is used (unless expressly specified otherwise) merely to indicate a particular feature, such as to distinguish that particular feature from another feature that is described by the same term or by a similar term. For example, a “first widget” may be so named merely to distinguish it from, e.g., a “second widget”. Thus, the mere usage of the ordinal numbers “first” and “second” before the term “widget” does not indicate any other relationship between the two widgets, and likewise does not indicate any other characteristics of either or both widgets. For example, the mere usage of the ordinal numbers “first” and “second” before the term “widget” (1) does not indicate that either widget comes before or after any other in order or location; (2) does not indicate that either widget occurs or acts before or after any other in time; and (3) does not indicate that either widget ranks above or below any other, as in importance or quality. In addition, the mere usage of ordinal numbers does not define a numerical limit to the features identified with the ordinal numbers. For example, the mere usage of the ordinal numbers “first” and “second” before the term “widget” does not indicate that there must be no more than two widgets.

When a single device or article is described herein, more than one device or article (whether or not they cooperate) may alternatively be used in place of the single device or article that is described. Accordingly, the functionality that is described as being possessed by a device may alternatively be possessed by more than one device or article (whether or not they cooperate).

Similarly, where more than one device or article is described herein (whether or not they cooperate), a single device or article may alternatively be used in place of the more than one device or article that is described. For example, a plurality of computer-based devices may be substituted with a single computer-based device. Accordingly, the various functionality that is described as being possessed by more than one device or article may alternatively be possessed by a single device or article.

The functionality and/or the features of a single device that is described may be alternatively embodied by one or more other devices which are described but are not explicitly described as having such functionality and/or features. Thus, other embodiments need not include the described device itself, but rather can include the one or more other devices which would, in those other embodiments, have such functionality/features.

Devices that are in communication with each other need not be in continuous communication with each other, unless expressly specified otherwise. On the contrary, such devices need only transmit to each other as necessary or desirable, and may actually refrain from exchanging data most of the time. For example, a machine in communication with another machine via the Internet may not transmit data to the other machine for weeks at a time. In addition, devices that are in communication with each other may communicate directly or indirectly through one or more intermediaries.

A description of an embodiment with several components or features does not imply that all or even any of such components and/or features are required. On the contrary, a variety of optional components are described to illustrate the wide variety of possible embodiments of the present invention(s). Unless otherwise specified explicitly, no component and/or feature is essential or required.

Further, although process steps, algorithms or the like may be described in a sequential order, such processes may be configured to work in different orders. In other words, any sequence or order of steps that may be explicitly described does not necessarily indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the invention, and does not imply that the illustrated process is preferred.

Although a process may be described as including a plurality of steps, that does not indicate that all or even any of the steps are essential or required. Various other embodiments within the scope of the described invention(s) include other processes that omit some or all of the described steps. Unless otherwise specified explicitly, no step is essential or required.

Although a product may be described as including a plurality of components, aspects, qualities, characteristics and/or features, that does not indicate that all of the plurality are essential or required. Various other embodiments within the scope of the described invention(s) include other products that omit some or all of the described plurality.

An enumerated list of items (which may or may not be numbered) does not imply that any or all of the items are mutually exclusive, unless expressly specified otherwise. Likewise, an enumerated list of items (which may or may not be numbered) does not imply that any or all of the items are comprehensive of any category, unless expressly specified otherwise. For example, the enumerated list “a computer, a laptop, a PDA” does not imply that any or all of the three items of that list are mutually exclusive and does not imply that any or all of the three items of that list are comprehensive of any category.

Headings of sections provided in this patent application and the title of this patent application are for convenience only, and are not to be taken as limiting the disclosure in any way.

“Determining” something can be performed in a variety of manners and therefore the term “determining” (and like terms) includes calculating, computing, deriving, looking up (e.g., in a table, database or data structure), ascertaining and the like

The terms “including”, “comprising” and variations thereof mean “including but not limited to”, unless expressly specified otherwise. As used herein, “comprising” means “including,” and the singular forms “a” or “an” or “the” include plural references unless the context clearly dictates otherwise. The term “or” refers to a single element of stated alternative elements or a combination of two or more elements, unless the context clearly indicates otherwise

A description of an embodiment with several components or features does not imply that all or even any of such components and/or features are required. On the contrary, a variety of optional components are described to illustrate the wide variety of possible embodiments of the present invention(s). Unless otherwise specified explicitly, no component and/or feature is essential or required.

Further, although process steps, algorithms or the like may be described in a sequential order, such processes may be configured to work in different orders. In other words, any sequence or order of steps that may be explicitly described does not necessarily indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the invention, and does not imply that the illustrated process is preferred.

The present disclosure provides, to one of ordinary skill in the art, an enabling description of several embodiments and/or inventions. Some of these embodiments and/or inventions may not be claimed in the present application, but may nevertheless be claimed in one or more continuing applications that claim the benefit of priority of the present application. Applicants intend to file additional applications to pursue patents for subject matter that has been disclosed and enabled but not claimed in the present application.

It will be understood that various modifications can be made to the embodiments of the present disclosure herein without departing from the scope thereof. Therefore, the above description should not be construed as limiting the disclosure, but merely as embodiments thereof. Those skilled in the art will envision other modifications within the scope of the invention as defined by the claims appended hereto.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods, as known to those of ordinary skill in the art. When directly and explicitly distinguishing embodiments from discussed prior art, the embodiment numbers are not approximates unless the word “about” is recited. Whenever “substantially,” “approximately,” “about,” or similar language is explicitly used in combination with a specific value, variations up to and including ten percent (10%) of that value are intended, unless explicitly stated otherwise.

Directions and other relative references may be used to facilitate discussion of the drawings and principles herein, but are not intended to be limiting. For example, certain terms may be used such as “inner,” “outer,”, “upper,” “lower,” “top,” “bottom,” “interior,” “exterior,” “left,” right,” “front,” “back,” “rear,” and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” part can become a “lower” part simply by turning the object over. Nevertheless, it is still the same part and the object remains the same.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### VI. Rules of Interpretation

Any of the features illustrated or described with respect to FIGS. 1-3 and Clauses 1-34 can be combined with any other features illustrated or described with respect to FIGS. 1-3 and Clauses 1-34 to provide systems, assemblies, kits, devices, methods, and embodiments not otherwise illustrated or specifically described herein. All features described herein are independent of one another and, except where structurally impossible, can be used in combination with any other feature described herein.

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A pre-filled medical delivery assembly comprising:
(i) a first blow-fill-seal (BFS) module having a first reservoir having a first fluid agent therein and a first neck with a first seal at an end thereof, the first neck being in fluid communication with the first reservoir;
(ii) a second BFS module having a second reservoir having a second fluid agent therein and a second neck with a second seal at an end thereof, the second neck being in fluid communication with the second reservoir;
(iii) a first connector having a first recess constructed to receive at least part of the first neck of the first BFS module and an outlet port in fluid communication with the first recess, the first recess having a first piercing element therein; and
(iv) a second connector having a second recess constructed to receive at least part of the second neck of the second BFS module and an inlet/outlet (I/O) port in fluid communication with the second recess, the second recess having a second piercing element therein,
wherein the first and second connectors are constructed to be coupled together via the outlet port and the I/O port, so as to provide a fluid path between the first and second reservoirs.

2. The pre-filled medical delivery assembly of claim 1, wherein (a) an end of the first connector having the outlet port has threads for coupling to the second connector, (b) an end of the second connector having the I/O port has threads for coupling to the first connector, or both (a) and (b).

3. The pre-filled medical delivery assembly of claim 1, wherein:
the first connector is disposed between the first BFS module and the second connector with respect to a longitudinal direction of the first BFS module;
the second connector is disposed between the second BFS module and the first connector with respect to a longitudinal direction of the second BFS module;
the first and second connectors are disposed between the first and second BFS modules with respect to a longitudinal direction of either the first or second BFS module; or
any combination of the foregoing.

4. The pre-filled medical delivery assembly of claim 1, wherein a first longitudinal direction of the first BFS module extends from the first reservoir toward the first seal, a second longitudinal direction of the second BFS module extends from the second reservoir toward the second seal, and the first and second longitudinal directions are co-axial.

5. The pre-filled medical delivery assembly of claim 4, wherein a central axis of the outlet port, a central axis of the I/O port, or both are co-axial with the first and second longitudinal directions.

6. The pre-filled medical delivery assembly of claim 1, wherein (c) the first piercing element breaches or extends through the first seal, (d) the second piercing element breaches or extends through the second seal, or both (c) and (d).

7. The pre-filled medical delivery assembly of claim 1, wherein (e) a tip of the first piercing element is spaced from the first seal, (f) a tip of the second piercing element is spaced from the second seal, or both (e) and (f).

8. The pre-filled medical delivery assembly of claim 1, wherein (g) an exterior of the first connector has a knurled surface, (h) an exterior of the second connector has a knurled surface, or both (g) and (h).

9. The pre-filled medical delivery assembly of claim 1, wherein the second connector comprises an annular hub surrounding the I/O port.

10. The pre-filled medical delivery assembly of claim 1, wherein:
(j) the first recess has a first mating feature, and the first neck has a laterally-protruding portion that is received in the first mating feature;
(k) the second recess has a second mating feature, and the second neck has a laterally-protruding portion that is received in the second mating feature; or
both (j) and (k).

11. The pre-filled medical delivery assembly of claim 10, wherein:
(l) with the laterally-protruding portion of the first neck received in the first mating feature, the first piercing element breaches or extends through the first seal;
(m) with the laterally-protruding portion of the second neck received in the second mating feature, the second piercing element breaches or extends through the second seal; or
both (l) and (m).

12. The pre-filled medical delivery assembly of claim 10, wherein:
(n) the first mating feature comprises a scalloped portion of a sidewall of the first recess, and the laterally-protruding portion of the first neck has a toroidal shape in a side view;
(o) the second mating feature comprises a scalloped portion of a sidewall of the second recess, and the laterally-protruding portion of the second neck has a toroidal shape in a side view; or
both (n) and (o).

13. The pre-filled medical delivery assembly of claim 1, wherein:
(p) a volume of the second reservoir is greater than a volume of the first reservoir;
(q) a volume of the second fluid agent is greater than a volume of the first fluid agent; or
both (p) and (q).

14. The pre-filled medical delivery assembly of claim 13, wherein the volume of the second reservoir is at least two times greater than the volume of the first reservoir.

15. The pre-filled medical delivery assembly of claim 1, wherein a volume of the second fluid agent is at least 2 ml, and a volume of the first fluid agent is about 0.7 ml.

16. The pre-filled medical delivery assembly of claim 1, wherein the first fluid agent, the second fluid agent, or both comprise a vaccine, a drug, a medicament, or a component of any of the foregoing.

17. The pre-filled medical delivery assembly of claim 1, wherein the first seal, the second seal, or both the first and second seals comprise a foil, wax, paper, a section of the respective BFS module, or any combination of the foregoing.

18. The pre-filled medical delivery assembly of claim 1, further comprising:
(v) an administration assembly comprising:
a hub constructed to be coupled to the I/O port of the second connector so as to provide a fluid path from the second reservoir; and
at least one administration conduit having an outlet at an end thereof longitudinally spaced from the hub and being in fluid communication with the second reservoir so as to receive a combination of the first and second fluid agents therefrom.

19. The pre-filled medical delivery assembly of claim 18, wherein the at least one administration conduit comprises a needle or cannula.

20. The pre-filled medical delivery assembly of claim 19, wherein the needle has a length in a range of 0.5 mm to 4 mm, inclusive, or in a range of 4 mm to 15 mm, inclusive, or in a range of 15 mm to 30 mm, inclusive.

21. The pre-filled medical delivery assembly of claim 18, wherein the outlet of the at least one administration conduit is formed as a nozzle configured to facilitate dispersion of the combination of the first and second fluid agents into a spray, or the outlet of the at least one administration conduit is formed as a nozzle configured to facilitate dispersion of the combination of the first and second fluid agents into one or more droplets.

22. The pre-filled medical delivery assembly of claim 1, wherein the first connector, the second connector, or both the first and second connectors are formed of a material having a hardness greater than that of the first BFS module, the second BFS module, or both the first and second BFS modules.
